(19) United States
(12) Patent Application Publication
(10) Pub. No.: US 2026/0224907 A1
(43) Pub. Date: Aug. 6, 2026

(54) LIGHT IRRADIATION DEVICE

(71) Applicant: TERUMO KABUSHIKI KAISHA, Tokyo (JP)

(72) Inventor: Satoru SUEHARA, Kaisei-machi (JP)

(73) Assignee: TERUMO KABUSHIKI KAISHA, Tokyo (JP)

(21) Appl. No.: 19/577,902

(22) Filed: Mar. 25, 2026

Related U.S. Application Data
(63) Continuation of application No. PCT/JP2024/033976, filed on Sep. 24, 2024.

(30) Foreign Application Priority Data
Sep. 28, 2023 (JP) 2023-167650

(51) Int. Cl. A61N 5/06; A61B 1/00

(52) U.S. Cl. CPC A61N 5/0603; A61B 1/00082; A61N 5/062; A61N 2005/0605; A61N 2005/0607; A61N 2005/0609

(57) ABSTRACT

A light irradiation device includes a balloon formed from a light transmissive material and a tubular body arranged therein and including a lumen through which a light irradiation body including a light irradiation unit is inserted. The balloon includes a light transmissive window portion that transmits light from the light irradiation unit and a light shielding body that has a lower light transmittance than the light transmissive window portion. The light transmissive window portion includes a first end portion on a distal side in a major axis direction of the balloon and a second end portion on a proximal side in the major axis direction of the balloon. The lumen includes a boundary portion on which a distal end of the light irradiation body iabuts at a position on the distal side of the first end portion and on the proximal side of a most distal end of the balloon.

### CROSS-REFERENCES TO RELATED APPLICATIONS

This application is a continuation of International Patent Application No. PCT/JP2024/033976 filed on September 24, 2024, which claims priority to Japanese Patent Application No. 2023-167650 filed on September 28, 2023, the entire content of both of which is incorporated herein by reference.

### TECHNOLOGICAL FIELD

The present invention relates generally to a light irradiation device to be used for treatment of irradiating a lesion with light.

### BACKGROUND DISCUSSION

As local treatment for cancer, photodynamic therapy (PDT) and photoimmunotherapy (PIT) to be performed using photoreactive substances having tumor cell selectivity are known. In this treatment, a light irradiation device that irradiates a lesion with light is required. A light irradiation device that performs light irradiation includes at least a light irradiation unit that emits light and an elongated member having the light irradiation unit at a distal end portion.

An example of an irradiation device that does not perform light irradiation but includes a radiation source that performs radiation irradiation and an elongated member having the radiation source at a distal end portion is disclosed in JP 2001-46532 A. This device is configured to have a lumen that opens at a distal end portion of a tubular body, allow a guidewire to penetrate the lumen, and prevent a radiation wire having the radiation source from penetrating the lumen.

### SUMMARY

In order to perform reliable treatment, a light irradiation device that performs light irradiation is required to locally approach a lesion and stably irradiate only the lesion and its surroundings with light necessary for treatment. As a light irradiation device for treating a lesion of a hollow organ such as a digestive organ, a device having a balloon at a distal end portion of an elongated member can be used. In this light irradiation device, a light irradiation body having a light irradiation unit is arranged inside the balloon, and light can be emitted from a light transmissive window portion disposed in the balloon. The light irradiation device can maintain a constant distance between the lesion and the light irradiation unit by expanding the balloon, and can fix a position of the light irradiation unit with respect to the lesion, so that the lesion can be stably irradiated with light. In the light irradiation device, the light irradiation body is inserted into a lumen of a tubular body disposed inside the elongated member. The light irradiation device irradiates the lesion with light from the light irradiation unit through the light transmissive window portion of the balloon, necessitating reliable positioning of the light irradiation body with respect to the balloon.

In addition, the light irradiation device having the balloon and capable of performing light irradiation can be delivered to the lesion using a lumen of an endoscope. When the light irradiation device is inserted into the lumen of the endoscope, the balloon is wrapped. However, a distal end portion of the light irradiation device has a large outer diameter due to the balloon and has a small clearance with an inner wall of the endoscope. Thus, in order to suppress deformation such as buckling of an elongated portion when the light irradiation device is inserted into the lumen of the endoscope, it is also required that a core wire (stylet) having a diameter different from that of the light irradiation body can be inserted into the lumen of the tubular body up to the distal end portion of the light irradiation device.

A light irradiation device disclosed here allows insertion of both a core wire and a light irradiation body and enables the light irradiation body to be reliably positioned with respect to a balloon.

A light irradiation device disclosed here includes: a balloon formed from a light transmissive material; and a tubular body arranged inside the balloon and including a lumen through which a light irradiation body including a light irradiation unit is inserted, in which the balloon includes a light transmissive window portion that transmits light from the light irradiation unit and a light shielding body that is disposed in a region other than the light transmissive window portion and has a lower transmittance of light from the light irradiation unit than a transmittance of the light transmissive window portion, the light transmissive window portion including a first end portion on a distal side in a major axis direction of the balloon and a second end portion on a proximal side in the major axis direction of the balloon, and the lumen of the tubular body includes a boundary portion on which a distal end of the light irradiation body abuts, at a position on the distal side of the first end portion and on the proximal side of a most distal end of the balloon.

The light irradiation device configured as described above allows both the light irradiation body for treatment and the core wire as a reinforcing body at the time of insertion to be inserted into the lumen of the tubular body. It is therefore possible to reliably and easily insert the light irradiation device into the endoscope by inserting the core wire into the lumen of the tubular body, and when the light irradiation body is inserted into the lumen of the tubular body instead of the core wire, it is possible to reliably position the light irradiation body with respect to the balloon and irradiate the lesion with light by making the distal end of the light irradiation body abut on the boundary portion.

In the light irradiation device, the lumen of the tubular body may include a small diameter portion on the distal side of the boundary portion and a large diameter portion having an inner diameter larger than an inner diameter of the small diameter portion on the proximal side of the boundary portion. As a result, the core wire can be inserted up to the distal end portion of the light irradiation device and can reliably support a portion up to a distal end of the light irradiation device, thereby the light irradiation device can be more reliably and easily inserted into the endoscope.

In the light irradiation device, the inner diameter of the small diameter portion may be smaller than an outer diameter of the light irradiation body. As a result, the light irradiation body cannot enter the small diameter portion, so that the distal end of the light irradiation body reliably abuts on the boundary portion, and positioning can be performed.

In the light irradiation device, the inner diameter of the small diameter portion may be larger than an outer diameter of a core wire to be inserted into the lumen. As a result, the core wire can be reliably inserted into the small diameter portion.

In the light irradiation device, the light irradiation unit may be disposed on the distal side in the major axis direction of the light irradiation body, and includes a distal end that may be located on the distal side of the first end portion, and a proximal end that may be located on the proximal side of the second end portion in a state in which the distal end of the light irradiation body abuts on the boundary portion. As a result, the light irradiation unit emits light over an entire length of the light transmissive window portion, so that light irradiation can be uniformly performed from the light transmissive window portion.

In the light irradiation device, a diameter of the boundary portion may continuously decrease from the large diameter portion toward the small diameter portion. As a result, the core wire can be easily inserted from the large diameter portion toward the small diameter portion.

In the light irradiation device, the tubular body may include an outer layer portion that covers an outer side of the small diameter portion. The resulting rigidity at the distal end portion of the tubular body can help suppress deflection, or the like, at the time of insertion of the light irradiation device.

In the light irradiation device, the tubular body may include an outer layer portion that integrally covers outer sides of the small diameter portion, the boundary portion, and the large diameter portion. The resulting rigidity at the distal end portion of the tubular body including the boundary portion can help suppress deflection, or the like, at the time of insertion of the light irradiation device.

In the light irradiation device, the tubular body may include a deformation absorption portion having an outer diameter smaller than an outer diameter of the large diameter portion at a proximal end portion. As a result, the deformation absorption portion is easily deformed at the time of insertion of the light irradiation device, so that it is possible to suppress occurrence of deflection, twisting, or the like, on the distal side of the tubular body.

### BRIEF DESCRIPTION OF THE DRAWINGS

FIG. 1 is an overall view of a light irradiation device and an endoscope according to the present embodiment;

FIG. 2 is a perspective view of a balloon;

FIG. 3 is an enlarged cross-sectional view of the vicinity of a distal end portion of the light irradiation device inserted into the endoscope;

FIG. 4 is an enlarged cross-sectional view of the vicinity of a fixing adapter and an operation handle of the light irradiation device inserted into the endoscope;

FIG. 5 is an enlarged cross-sectional view of the vicinity of a hub of the light irradiation device;

FIG. 6 is a flowchart of procedure using the light irradiation device; and

FIG. 7 is an enlarged cross-sectional view of the vicinity of the distal end portion of the light irradiation device in a state in which a core wire is inserted into a tubular body.

### DETAILED DESCRIPTION

Hereinafter, an embodiment of the light irradiation device, representing examples of the new light irradiation device disclosed here will be described with reference to the drawings. Note that, the dimensions of the drawings may be exaggerated and different from actual dimensions for convenience of description in some cases. In the present specification and the drawings, components having substantially the same functional configuration are denoted by the same reference numerals, and the redundant description will not be repeated. In the present specification, a side of a device to be inserted into a living body is referred to as a “distal side”, and a side to be operated is referred to as a “proximal side”.

A light irradiation device 10 according to the present embodiment is used for treatment of a tumor. A type of the tumor is not particularly limited, but for example, in a case where the light irradiation device 10 is inserted into the endoscope 100 and used as an endoscope system, the light irradiation device 10 can be applied to a tumor generated in a hollow organ such as the esophagus, the stomach, the small intestine, the large intestine, the urinary tract, a blood vessel, the ear canal, the auditory tube, and the nasal cavity. The light irradiation device 10 in the present embodiment is used, for example, in photoimmunotherapy taught in Japanese Patent No. 6127045 in which a drug adsorbed to a target cell is irradiated with light to destroy the target cell. The target cell is a tumor cell such as a cancer cell or a cell of a precancerous lesion. In this therapeutic method, a photosensitizer in which an antibody that specifically binds to only a specific antigen on the surface of a tumor cell and a photosensitizer paired with the antibody are adsorbed is used as a drug. The antibody is not particularly limited, and examples thereof include panitumumab, trastuzumab, HuJ591, pertuzumab, lapatinib, palbociclib, olaparib, and the like. The photosensitizer is, for example, but not limited to, hydrophilic phthalocyanine, which is a substance reactive to near infrared rays having a wavelength of about 700 nm (IR700). When IR700 receives near infrared rays having a wavelength of about in a range of 660 to 740 nm, a ligand of a functional group that ensures water solubility is broken, and a structural change from water solubility to hydrophobicity occurs. The membrane protein is extracted by this structural change, a hole is formed in the cell membrane, and water enters the cell, whereby the tumor cell can be ruptured and destroyed. In addition, the IR700 is excited by receiving near infrared rays and emits fluorescence having a wavelength different from the excitation wavelength. For example, when the IR700 is excited by receiving near infrared rays having a wavelength around 690 nm, the IR700 emits fluorescence having a wavelength around 700 nm. The IR700 undergoes a structural change while emitting fluorescence by photoreaction, and does not emit fluorescence when it destroys tumor cells and serves as a drug.

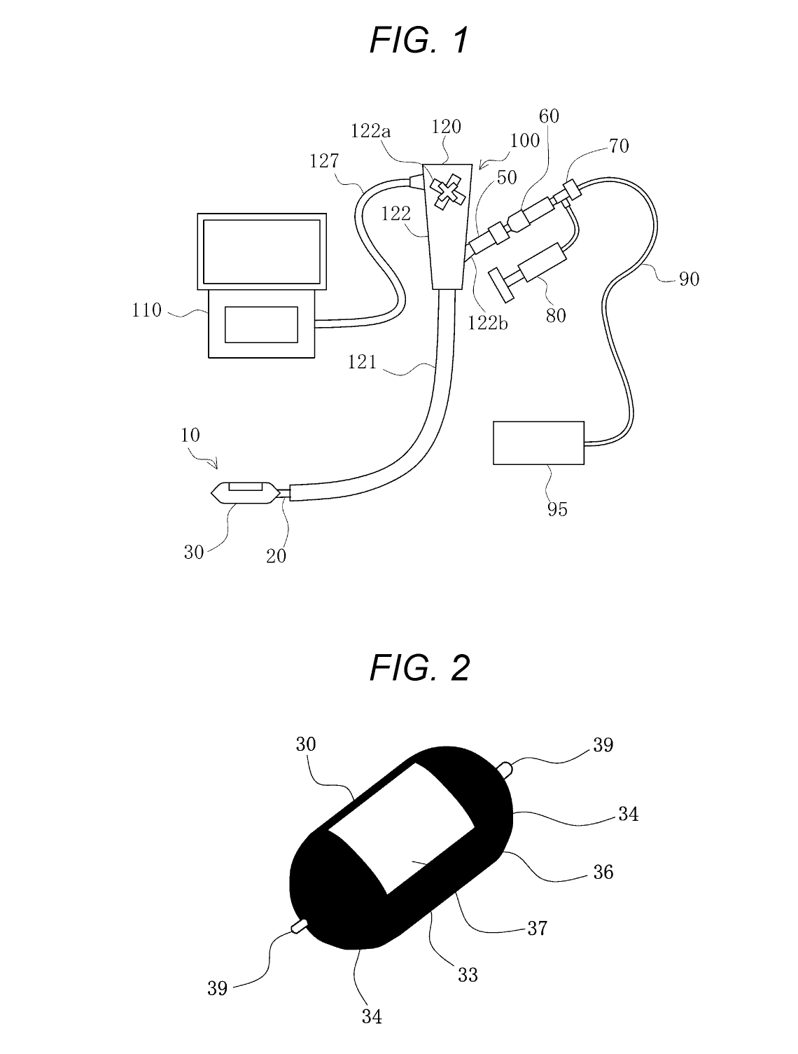

The light irradiation device 10 of the present embodiment can be configured to be compatible with treatment that approaches tumor cells via an endoscope 100. As illustrated in FIG. 1, the light irradiation device 10 is used by being inserted into the endoscope 100, for example.

The endoscope 100 includes an endoscope scope 120 connected to a control display unit 110 via a connection line 127. An operator inserts the endoscope scope 120 into a living body and performs various kinds of operation. The endoscope scope 120 includes an elongated insertion portion 121 to be inserted into the living body and a handle portion 122 disposed at a proximal end portion of the elongated insertion portion 121.

The handle portion 122 includes an operation portion 122a for the operator to operate bending of the elongated insertion portion 121, air supply, water supply, suction, and the like, and a forceps port 122b communicating with a lumen 125 of the elongated insertion portion 121. The light irradiation device 10 is inserted into the endoscope 100 from the forceps port 122b of the handle portion 122.

The light irradiation device 10 includes an elongated shaft portion 20 to be inserted into the endoscope 100 and a balloon 30 disposed at a distal end portion of the shaft portion 20 and exposed to a distal side of the elongated insertion portion 121. Further, the light irradiation device 10 includes a fixing adapter 50 to be fixed to the forceps port 122b, an operation handle 60 to be used for positioning the balloon 30, and a hub 70 disposed at a proximal end portion of the light irradiation device 10. An expansion device 80 for injecting a fluid for expanding the balloon 30 is connected to the hub 70. As the expansion device 80, for example, an indeflator can be used.

A light irradiation body 90 that emits light in the balloon 30 is inserted into the light irradiation device 10. The light irradiation body 90 is exposed to the proximal side of the hub 70 of the light irradiation device 10 and is connected to a light source unit 95 that outputs light. The light irradiation body 90 includes a light irradiation unit 92 disposed on the distal side in a major axis direction of the light irradiation body 90 and an optical fiber connected to the light irradiation unit 92 and extending toward the proximal side in the major axis direction of the light irradiation body 90. For example, the light irradiation body 90 is comprised of a side emission fiber capable of irradiating an entire circumference on the outer side in a radial direction with light at a distal end portion. The light irradiation unit 92 and the optical fiber can be configured so that a diameter of the light irradiation unit 92 is larger than a diameter of the optical fiber in the major axis direction of the light irradiation body 90.

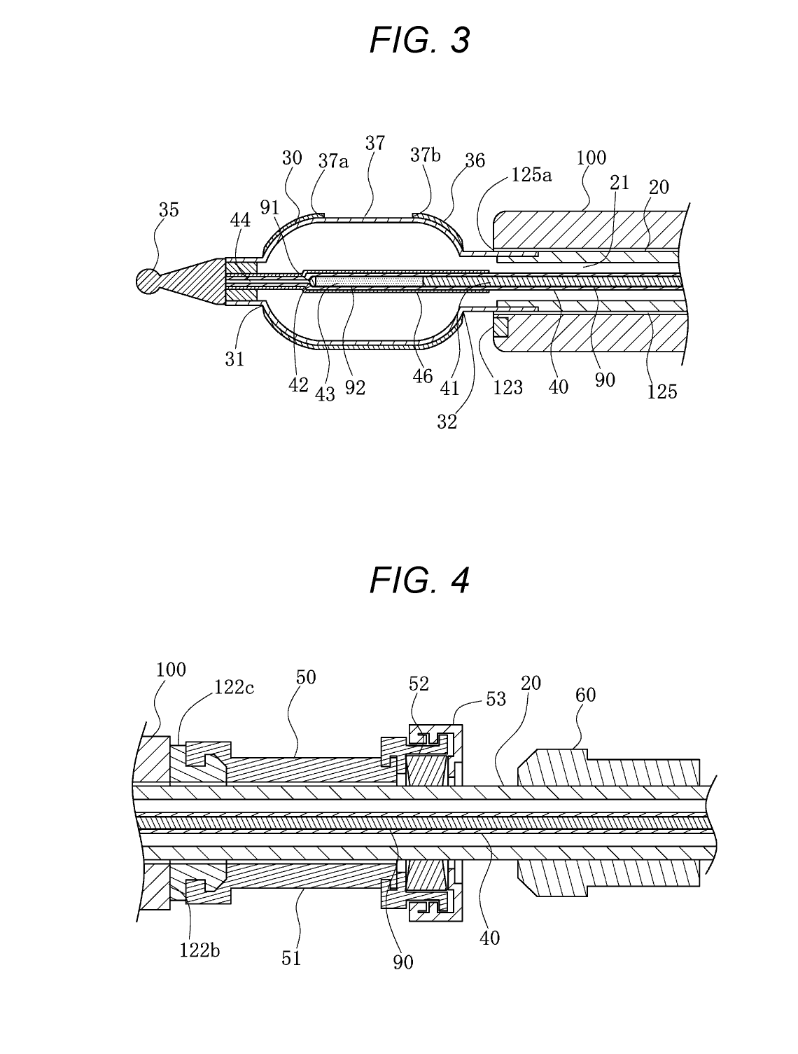

As illustrated in FIGS. 2 and 3, the balloon 30 may be radially expanded. As illustrated in FIG. 3, the balloon 30 is a portion configured to be expanded in the radial direction between a most distal end 31 and a most proximal end 32. A central portion in the major axis direction of the balloon 30 is a straight portion 33 having the same diameter along the major axis direction, and both end portions in the major axis direction of the balloon 30 are tapered portions 34 having diameters decreasing toward both ends. As illustrated in FIG. 2, the tapered portions 34 can be formed in a hemispherical shape. However, the shape of the tapered portions 34 is not limited to a hemispherical shape, and may be, for example, a conical shape. The most distal end in the major axis direction of the balloon 30 (the straight portion 33 and the tapered portion 34) configured to be expanded and deflated in the radial direction is defined as the most distal end 31, and the most proximal end in the major axis direction is defined as the most proximal end 32. The balloon 30 is formed from a light transmissive material such as nylon or urethane. The balloon 30 is formed by blow molding a parison formed from these light transmissive materials, and tubular portions 39 as a margin at the time of molding remain at the distal end from the most distal end 31 and at the proximal end from the most proximal end 32 of the balloon 30. These tubular portions 39 are configured not to be expanded or deflated in the radial direction, and are used as fixing portions with respect to a shaft portion 20 and a tubular body 40 described later.

The light irradiation body 90 is arranged inside the balloon 30. The balloon 30 includes a light transmissive window portion 37 that transmits light from the light irradiation unit 92 included in the light irradiation body 90, and a light shielding body 36 having a lower transmittance of light from the light irradiation unit 92 than a transmittance of the light transmissive window portion 37. The light shielding body 36 can be ink, a metal film, or the like, that covers a surface of the balloon 30. In other words, the balloon 30 includes, as the light shielding body 36, a membrane body formed by a coating material coated on an outer surface of the balloon 30. The surface of the balloon 30 is not covered with the light shielding body 36, and thus, the light transmissive window portion 37 can transmit light from the light irradiation unit 92. In other words, a portion not covered with the light shielding body 36 is defined as the light transmissive window portion 37. The light transmissive window portion 37 may be disposed over the entire circumferential direction (360 degrees) or may be disposed in part of the circumferential direction (for example, 180 degrees).

As illustrated in FIG. 3, the balloon 30 can be expanded by being exposed to the distal side from a distal opening 125a of the lumen 125 of the endoscope 100. The endoscope 100 includes an endoscope objective lens 123 at a distal end portion, and a state of the balloon 30 can be visually recognized. A proximal end portion of the balloon 30 is bonded to a distal end portion of the shaft portion 20 inserted into the lumen 125 of the endoscope 100. A distal end tip 35 is disposed at a distal end portion of the balloon 30. The light transmissive window portion 37 of the balloon 30 has a first end portion 37a on the distal side in the major axis direction of the balloon 30 and a second end portion 37b on the proximal side in the major axis direction of the balloon 30.

The tubular body 40 having the lumen 41 along the major axis direction is arranged inside the balloon 30. The tubular body 40 extends along the major axis direction inside the shaft portion 20. A portion inside the shaft portion 20 and outside the tubular body 40 is an expansion lumen 21 through which a fluid for expanding the balloon 30 flows. A distal end portion of the tubular body 40 extends beyond the most distal end 31 of the balloon 30 and is fixed to the balloon 30.

The light irradiation body 90 is inserted into the lumen 41 of the tubular body 40. The light irradiation body 90, which is a side emission fiber from which light is emitted to the entire circumference on the outer side in the radial direction, has a light irradiation unit 92 that can perform light irradiation with uniform intensity in the radial direction along the major axis direction at the distal end portion. In the tubular body 40, at least a portion located inside the balloon 30 is formed from a light transmissive material in order to transmit light from the light irradiation unit 92.

The lumen 41 of the tubular body 40 has a stepped boundary portion 42 having a large inner diameter on the proximal side and a small inner diameter on the distal side at a position on the distal side of the first end portion 37a of the light transmissive window portion 37 and on the proximal side of the most distal end 31 of the balloon 30. The lumen 41 of the tubular body 40 has a small diameter portion 44 on the distal side of the boundary portion 42 and a large diameter portion 43 having an inner diameter larger than that of the small diameter portion 44 on the proximal side of the boundary portion 42. The large diameter portion 43 has an inner diameter larger than the outer diameter of the light irradiation body 90 and allows insertion of the light irradiation body 90. The small diameter portion 44 has an inner diameter smaller than the outer diameter of the light irradiation body 90 and does not allow insertion of the light irradiation body 90. Thus, as a result of the distal end 91 being made to abut on the boundary portion 42, the light irradiation body 90 cannot move further to the distal side, and the light irradiation unit 92 can be positioned at a certain position inside the balloon 30.

In a state in which the distal end 91 of the light irradiation body 90 abuts on the boundary portion 42, the distal end of the light irradiation unit 92 is located on the distal side of the first end portion 37a of the light transmissive window portion 37, and the proximal end is located on the proximal side of the second end portion 37b of the light transmissive window portion 37. As a result, the light irradiation unit 92 can be reliably positioned in the entire range in the major axis direction of the light transmissive window portion 37, and irradiation of light directed in the radial direction from the light irradiation unit 92 is uniformly performed from the inside of the balloon 30 through the light transmissive window portion 37.

The boundary portion 42 continuously decreases in diameter from the large diameter portion 43 toward the small diameter portion 44. Thus, a discontinuous corner does not occur in the boundary portion 42, and insertion of the core wire 200 described later can be facilitated. The small diameter portion 44 having a diameter decreasing from the large diameter portion 43 via the boundary portion 42 is arranged coaxially with the large diameter portion 43. This also facilitates insertion of the core wire 200 to be described later.

The outer diameter of the tubular body 40 also decreases from the large diameter portion 43 toward the small diameter portion 44. The tubular body 40 may have an outer layer portion 46 that integrally covers outer sides of the small diameter portion 44, the boundary portion 42, and the large diameter portion 43. The outer layer portion 46 is only required to be formed so as to cover at least the small diameter portion 44, or the outer layer portion 46 may be configured to cover the small diameter portion 44 and the boundary portion 42. The outer layer portion 46 covers the tubular body 40 including a region where the light irradiation unit 92 of the light irradiation body 90 is arranged, and thus, is formed from a light transmissive material. The outer layer portion 46 can reinforce strength of the tubular body 40 that is decreased due to a step formed at the position of the boundary portion 42, and can facilitate insertion of the light irradiation device 10. The outer layer portion 46 may cover only the small diameter portion 44. Alternatively, the outer layer portion 46 may cover only the small diameter portion 44 and the boundary portion 42. In these cases, the outer layer portion 46 does not include the region where the light irradiation unit 92 is arranged, and thus, may be formed from a material other than the light transmissive material. The outer layer portion 46 can reinforce the small diameter portion 44 in which the strength is further decreased due to the smaller outer diameter, and when the balloon 30 is folded and wrapped, the outer diameter after wrapping can be prevented from becoming larger, and the light irradiation device 10 can be more reliably and easily inserted using the endoscope 100. As a method for reinforcing strength of the tubular body 40 that is decreased due to the step formed at the position of the boundary portion 42, a method in which a step is formed by inserting a tubular body smaller than the inner diameter of the tubular body 40 on the distal side of the lumen of the tubular body 40 may be selected. In this case, it is possible to form the step at the position of the boundary portion 42 without changing the outer diameter of the tubular body 40 over the entire range in the major axis direction while maintaining rigidity of the tubular body 40.

The endoscope scope 120 of the endoscope 100 has a total length of 1350 mm, and an effective length of the elongated insertion portion 121 is 1030 mm. The light irradiation device 10 has a total length in a range of 1600 mm to 1800 mm. The light irradiation body 90 has a total length of 2000 mm or more. However, these lengths are merely examples, and the lengths of the endoscope 100 and the light irradiation device 10 may be out of these ranges.

In the balloon 30, the length of the straight portion 33 in the major axis direction is in a range of 30 mm to 60 mm, and the outer diameter of the straight portion 33 at the time of expansion is in a range of 10 to 25 mm. The balloon 30 has a film thickness of about 0.03 mm, and the light shielding body 36 has a thickness of about from 0.006 to 0.03 mm. However, these dimensions are merely examples, and the length and thickness of the balloon 30 may be out of these ranges.

As illustrated in FIG. 4, a forceps port member 122c is arranged at the forceps port 122b of the endoscope 100. The fixing adapter 50 of the light irradiation device 10 includes an endoscope fixing portion 51 to be fitted and fixed to the forceps port member 122c. In other words, the forceps port member 122c serves as an interface for fitting and fixing the fixing adapter 50 to the forceps port 122b such that the interior of the fixing adapter 50 is in communication with the interior of the endoscope 100 via the forceps port 122b. The endoscope fixing portion 51 has a tubular shape and allows insertion of the shaft portion 20. The fixing adapter 50 has a fixing member 52 having flexibility at a proximal end portion. The fixing member 52 is formed in a ring shape surrounding an outer periphery of the shaft portion 20. Further, a rotating portion 53 that abuts on the fixing member 52 is disposed at the proximal end portion of the fixing adapter 50. By rotating the rotating portion 53, the rotating portion 53 moves to the distal side of the fixing adapter 50 and compresses the fixing member 52 on which the rotating portion 53 abuts in a thickness direction (major axis direction). The compressed fixing member 52 is deformed so that an inner diameter becomes smaller, and the shaft portion 20 can be fixed to the fixing adapter 50.

The operation handle 60 is located on the proximal side of a portion to be fixed to the fixing adapter 50, in the shaft portion 20, and can be gripped by the operator to move the shaft portion 20 in the major axis direction and can be rotated in the circumferential direction. As a result, a major axis direction position and a circumferential direction position of the balloon 30 can be adjusted after the light irradiation device 10 is inserted into the endoscope 100.

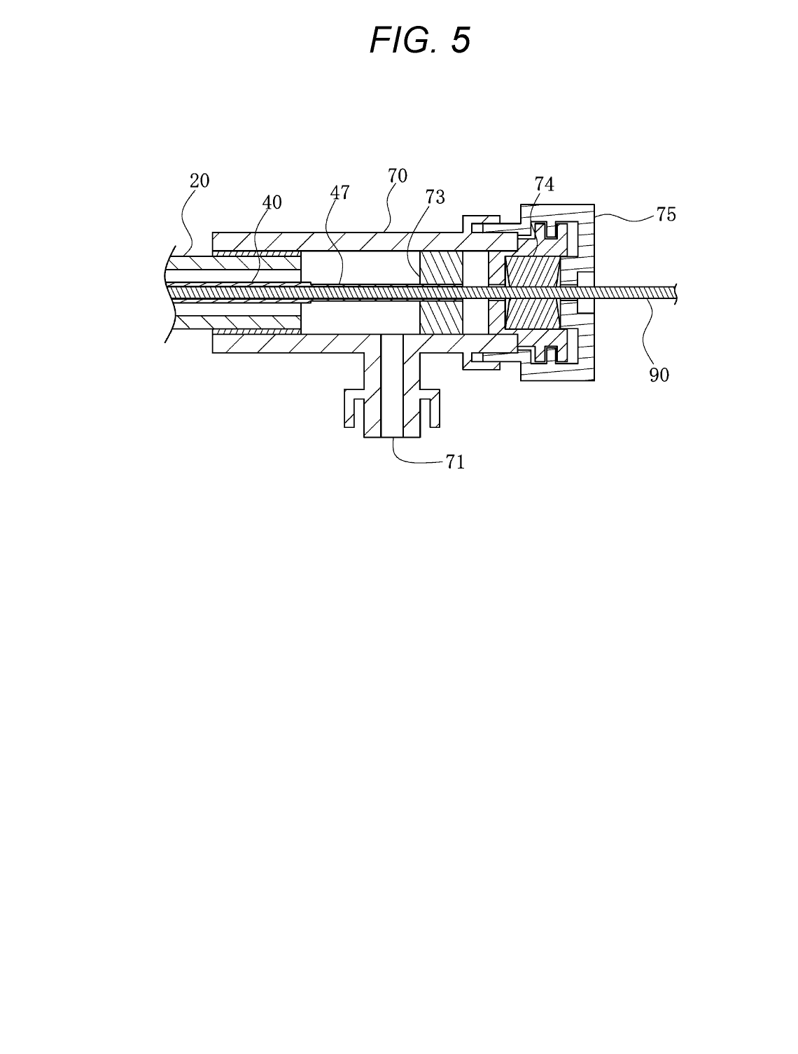

As illustrated in FIG. 5, the hub 70 has a hollow interior, and the hollow interior communicates with a connection port (side port) 71 to which the expansion device 80 can be connected. A proximal end portion of the shaft portion 20 is bonded and fixed to the inside of the hub 70. The proximal end portion of the tubular body 40 extends to the proximal side from the proximal end of the shaft portion 20 inside the hub 70 and is fixed at the tubular body fixing portion 73.

The tubular body 40 includes a deformation absorption portion 47 having an outer diameter smaller than that of the large diameter portion 43, at the proximal end portion. As a result of the tubular body 40 including the deformation absorption portion 47, in a case where the tubular body 40 receives an external force when the light irradiation device 10 is inserted into the endoscope 100, the deformation absorption portion 47 having a small wall thickness and being easily deformed is deformed, so that it is possible to suppress deflection or twisting of the portion on the distal side of the tubular body 40.

The light irradiation body 90 extends from the proximal end to the proximal side of the tubular body 40 and penetrates the hub 70. A light irradiation device fixing member 74 is disposed at the proximal end portion of the hub 70 so as to surround the light irradiation body 90. Further, a rotating portion 75 is disposed at the proximal end portion of the hub 70. By rotating the rotating portion 75, the rotating portion 75 moves to the distal side of the hub 70 to compress the light irradiation device fixing member 74, and an inner diameter of the light irradiation device fixing member 74 becomes small, so that the light irradiation body 90 can be fixed.

Next, a treatment method using the light irradiation device 10 will be described. Prior to treatment by the light irradiation device 10, a photosensitizer is administered into the body. A method for administering the photosensitizer into the body is not particularly limited as long as the photosensitizer can reach tumor cells, but is, for example, intravascular administration, and is intravenous administration in the present embodiment. After about 12 to 36 hours from the intravenous administration, treatment of light irradiation by the light irradiation device 10 of the present embodiment is performed.

As described above, the light irradiation device 10 approaches the lesion by being inserted into the endoscope 100. Thus, the endoscope 100 is inserted into the living body in advance before the treatment by the light irradiation device 10.

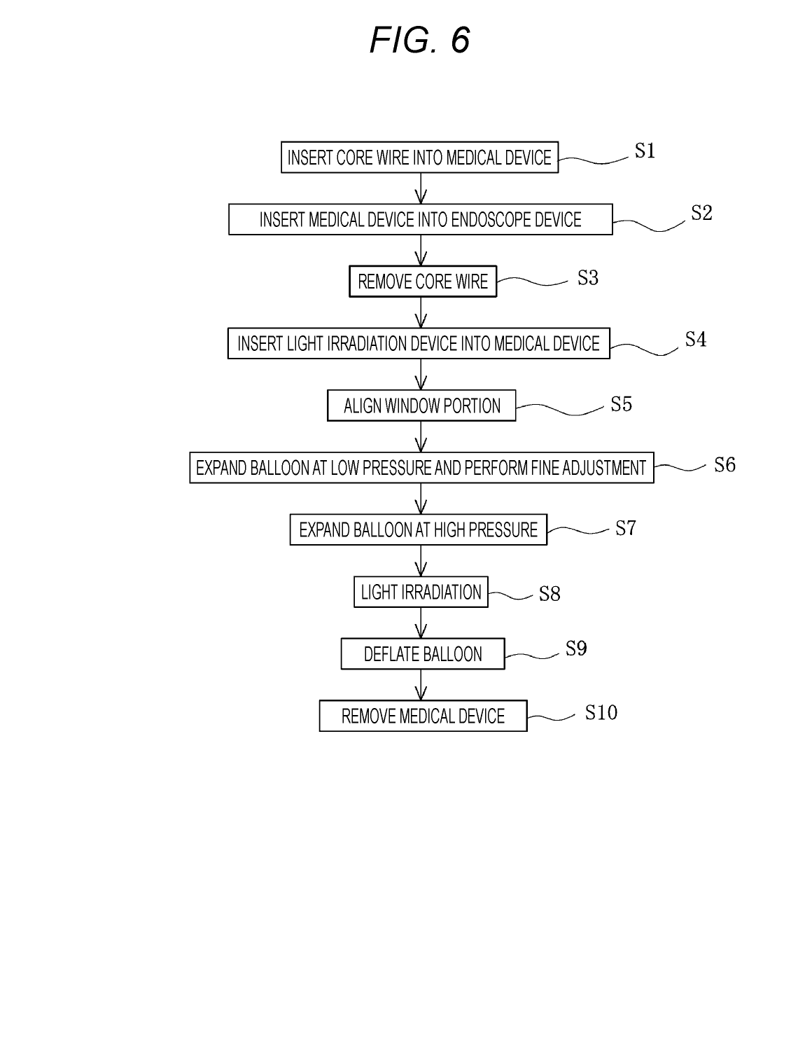

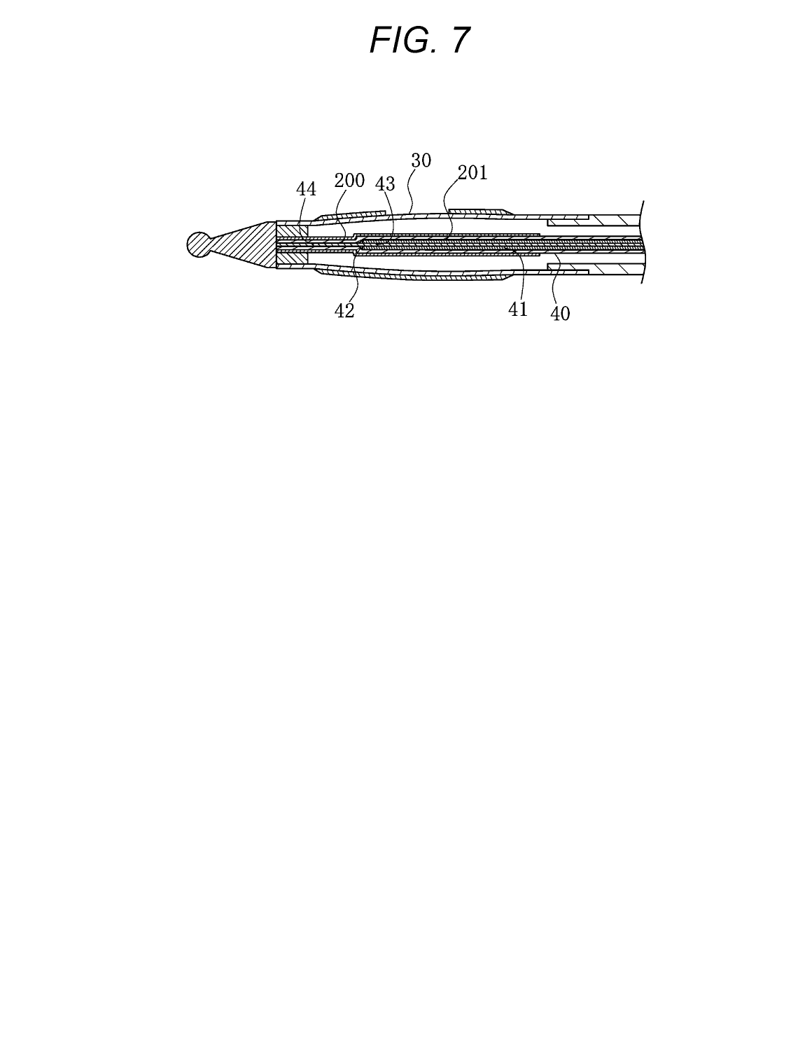

As indicated in FIG. 6, before the light irradiation device 10 is inserted into the endoscope 100, the core wire 200 is inserted into the light irradiation device 10 (S1). As illustrated in FIG. 7, the core wire 200 is inserted into the lumen 41 of the tubular body 40. The balloon 30 of the light irradiation device 10 before being inserted into the endoscope 100 is folded and wrapped so as to have a diameter that allows insertion into the lumen 125.

A portion of the core wire 200 other than the distal end portion is covered with a cover portion 201, and has an outer diameter close to the inner diameter of the large diameter portion 43 of the lumen 41. The cover portion 201 is not disposed at the distal end portion of the core wire 200, and the distal end portion of the core wire 200 has an outer diameter close to the inner diameter of the small diameter portion 44 of the lumen 41. As a result, the core wire 200 can be inserted over substantially the entire length from the large diameter portion 43 to the small diameter portion 44 of the lumen 41. In other words, the core wire 200 can be inserted up to the distal side of the light irradiation device 10 beyond the most distal end 31 of the balloon 30. The boundary portion 42 of the tubular body 40 is formed so that a diameter continuously decreases from the large diameter portion 43 toward the small diameter portion 44 as described above, and thus, it is easy to insert the core wire 200 toward the small diameter portion 44.

The core wire 200 can be formed from a metal such as stainless steel. Further, as the cover portion 201, a heat shrinkable tube formed from a resin, or the like, can be used. By inserting the core wire 200 into the lumen 41 of the tubular body 40, the resulting rigidity permits the light irradiation device 10 to be inserted into the endoscope 100 in a smooth manner. Furthermore, the core wire 200 can be inserted up to the small diameter portion 44, and thus, rigidity can be secured up to the distal end of the device, and the light irradiation device 10 can be inserted more reliably and easily. In the core wire 200, the distal end portion and the cover portion 201 may be integrally formed. Further, the core wire 200 does not have to be covered with the cover portion 201. In addition, the core wire 200 may be inserted until it enters and abuts on an innermost portion of the small diameter portion 44, or may be inserted until the cover portion 201 of the core wire 200 abuts on the boundary portion 42 of the tubular body 40 in a state in which the core wire 200 enters at least part of the small diameter portion 44. In other words, a length of the distal end portion of the core wire 200 and a length of the small diameter portion 44 may coincide with each other or does not have to coincide with each other.

After the core wire 200 is inserted until it abuts on the small diameter portion 44 of the light irradiation device 10, the light irradiation device 10 is inserted into the endoscope 100 (S2). The light irradiation device 10 is inserted into the lumen 125 from the fixing adapter 50 fixed to the forceps port 122b of the endoscope 100. The light irradiation device 10 is inserted until the balloon 30 is exposed from the distal opening 125a of the lumen 125.

After the light irradiation device 10 is inserted into the endoscope 100, the core wire 200 is removed from the tubular body 40 (S3). Next, the operator inserts the light irradiation body 90 into the light irradiation device 10 instead of the core wire 200 (S4). The light irradiation body 90 is inserted into the lumen 41 of the tubular body 40 from which the core wire 200 has been removed. The light irradiation body 90 is inserted until the distal end 91 abuts on the boundary portion 42 of the lumen 41. As a result, the light irradiation unit 92 of the light irradiation body 90 is positioned at a predetermined position in the balloon 30 (a position where the light irradiation unit 92 extends over the entire length of the light transmissive window portion 37). After the light irradiation body 90 is inserted and positioned with respect to the balloon 30, the operator rotates the rotating portion 75 of the hub 70 and fixes the light irradiation body 90 to the light irradiation device 10 by the light irradiation device fixing member 74.

Next, the operator grips the operation handle 60 and performs alignment while visually recognizing a marker of the light irradiation device 10 (not illustrated) and the lesion so that the light transmissive window portion 37 of the balloon 30 faces the direction of the lesion (S5). The operator can roughly perform alignment by adjusting the major axis direction position and the circumferential direction position of the light transmissive window portion 37 by the operation handle 60.

Next, the operator injects a fluid into the balloon 30 by the expansion device 80 and temporarily expands the balloon 30 at a low pressure to finely adjust a position of the balloon (S6). In S6, an expansion pressure of the balloon 30 may be set at 0.5 atm. However, the expansion pressure is not limited thereto. After the position of the balloon 30 is adjusted, the operator rotates the rotating portion 55 of the fixing adapter 50 to fix the light irradiation device 10 to the endoscope 100.

After finely adjusting the position of the balloon, the operator injects a fluid into the balloon 30 by the expansion device 80 to expand the balloon at a high pressure (S7). In S7, the expansion pressure of the balloon 30 may be set at 2 atm. However, the expansion pressure is not limited thereto.

When the balloon 30 is expanded at the lesion, the operator irradiates the lesion with light from the light irradiation unit 92 of the light irradiation body 90 (S8). The lesion is irradiated with light from the light irradiation unit 92 through the light transmissive window portion 37 of the balloon 30. The balloon 30 is expanded to be in contact with the hollow organ and fixed in the living body, so that it is possible to maintain a constant distance between the light irradiation unit 92 and the lesion and to stably supply necessary light energy to the lesion. In addition, light irradiation is performed through the light transmissive window portion 37 by the balloon 30, so that it is possible to irradiate only a necessary portion with light, and it is possible to reduce irradiation of normal tissues with light. The light irradiation is performed for a certain period of time, for example, 20 minutes. The light irradiation period is not limited to 20 minutes, and is set as necessary.

After the light irradiation is completed, the operator deflates the balloon 30 (S9) and removes the light irradiation device 10 from the endoscope 100 (S10).

As described above, the light irradiation device 10 according to the present embodiment includes the balloon 30 formed from a light transmissive material, and the tubular body 40 arranged inside the balloon 30 and including the lumen 41 through which the light irradiation body 90 including the light irradiation unit 92 is inserted, the balloon 30 includes the light transmissive window portion 37 that transmits the light from the light irradiation unit 92 and the light shielding body 36 that is disposed in a region other than the light transmissive window portion 37 and has a lower transmittance of light from the light irradiation unit 92 than a transmittance of the light transmissive window portion 37, the light transmissive window portion 37 including the first end portion 37a on a distal side in a major axis direction of the balloon 30 and the second end portion 37b on a proximal side in the major axis direction of the balloon 30, and the lumen 41 of the tubular body 40 includes the boundary portion 42 on which the distal end 91 of the light irradiation body 90 abuts, at a position on the distal side of the first end portion 37a and on the proximal side of the most distal end of the balloon 30. In the light irradiation device 10 configured as described above, both the light irradiation body 90 for treatment and the core wire 200 as a reinforcing body at the time of insertion can be inserted into the lumen 41 of the tubular body 40. Thus, the light irradiation device 10 can be reliably and easily inserted into the endoscope 100 by inserting the core wire 200 into the lumen 41 of the tubular body 40, and when the light irradiation body 90 is inserted into the lumen 41 of the tubular body 40 instead of the core wire 200, by making the distal end 91 of the light irradiation body 90 abut on the boundary portion 42, the light irradiation body 90 can be reliably positioned with respect to the balloon 30 to irradiate the lesion with light.

(2) In the light irradiation device 10 according to (1), the lumen 41 of the tubular body 40 may include the small diameter portion 44 on the distal side of the boundary portion 42, and the large diameter portion 43 having an inner diameter larger than an inner diameter of the small diameter portion 44 on the proximal side of the boundary portion 42. As a result, the core wire 200 can be inserted further up to the distal side beyond the distal end portion of the balloon 30 and can reliably support a portion up to the distal end of the light irradiation device 10, thereby the light irradiation device 10 can be more reliably and easily inserted into the endoscope 100.

(3) In the light irradiation device 10 according to (2), the inner diameter of the small diameter portion 44 may be smaller than the outer diameter of the light irradiation body 90. As a result, the light irradiation body 90 cannot enter the small diameter portion 44, and thus, the distal end of the light irradiation body 90 reliably abuts on the boundary portion 42, thereby positioning can be performed.

(4) In the light irradiation device 10 according to (2) or (3), the inner diameter of the small diameter portion 44 may be larger than an outer diameter of the core wire 200 to be inserted into the lumen 41. As a result, the core wire 200 can be reliably inserted into the small diameter portion 44.

(5) In the light irradiation device 10 according to any one of (1) to (4), the light irradiation unit 92 may be disposed on the distal side in the major axis direction of the light irradiation body 90, and includes the distal end that may be located on the distal side of the first end portion 37a, and the proximal end that may be located on the proximal side of the second end portion 37b in a state in which the distal end of the light irradiation body 90 abuts on the boundary portion 42. As a result, the light irradiation unit 92 emits light over the entire length of the light transmissive window portion 37, and thus, light irradiation can be uniformly performed from the light transmissive window portion 37.

(6) In the light irradiation device 10 according to any one of (2) to (5), a diameter of the boundary portion 42 may continuously decrease from the large diameter portion 43 toward the small diameter portion 44. As a result, the core wire 200 can be easily inserted from the large diameter portion 43 toward the small diameter portion 44.

(7) In the light irradiation device 10 according to any one of (2) to (6), the tubular body 40 may include the outer layer portion 46 that covers an outer side of the small diameter portion 44. As a result, rigidity at the distal end portion of the tubular body 40 can be secured, and deflection, or the like, at the time of insertion of the light irradiation device 10 can be suppressed.

(8) In the light irradiation device 10 according to any one of (2) to (6), the tubular body 40 may include the outer layer portion 46 integrally covering outer sides of the small diameter portion 44, the boundary portion 42, and the large diameter portion 43. As a result, rigidity at the distal end portion of the tubular body 40 including the boundary portion 42 having a step is secured, and deflection, or the like, at the time of insertion of the light irradiation device 10 can be suppressed.

(9) In the light irradiation device 10 according to any one of (2) to (8), the tubular body 40 may include the deformation absorption portion 47 having an outer diameter smaller than an outer diameter of the large diameter portion 43 at a proximal end portion. As a result, the deformation absorption portion 47 is easily deformed when the light irradiation device 10 is inserted, so that it is possible to suppress occurrence of deflection, twisting, or the like, on the distal side of the tubular body 40.

The detailed description above describes embodiments of a light irradiation representing examples of the new light irradiation device having an expansion body disclosed here. The invention is not limited, however, to the precise embodiment and modifications described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims. For example, in the above-described embodiment, the lumen 41 of the tubular body 40 has the small diameter portion 44 on the distal side of the boundary portion 42, but the distal side of the boundary portion 42 of the tubular body 40 may be solid.

What is claimed is:

1. A light irradiation device comprising: a balloon formed from a light transmissive material; and a tubular body arranged inside the balloon and including a lumen through which a light irradiation body including a light irradiation unit is inserted, wherein the balloon includes a light transmissive window portion that transmits light from the light irradiation unit and a light shielding body that is disposed in a region other than the light transmissive window portion and has a lower transmittance of light from the light irradiation unit than a transmittance of the light transmissive window portion, the light transmissive window portion including a first end portion on a distal side in a major axis direction of the balloon and a second end portion on a proximal side in the major axis direction of the balloon, and the lumen of the tubular body includes a boundary portion on which a distal end of the light irradiation body abuts, at a position on the distal side of the first end portion and on the proximal side of a most distal end of the balloon.

2. The light irradiation device according to claim 1, wherein the lumen of the tubular body includes a small diameter portion on the distal side of the boundary portion, and a large diameter portion having an inner diameter larger than an inner diameter of the small diameter portion on the proximal side of the boundary portion.

3. The light irradiation device according to claim 2, wherein the inner diameter of the small diameter portion is smaller than an outer diameter of the light irradiation body.

4. The light irradiation device according to claim 3, wherein the inner diameter of the small diameter portion is larger than an outer diameter of a core wire to be inserted into the lumen.

5. The light irradiation device according to claim 1, wherein the light irradiation unit is disposed on the distal side in the major axis direction of the light irradiation body, and includes a distal end located on the distal side of the first end portion, and a proximal end located on the proximal side of the second end portion in a state in which a distal end of the light irradiation body abuts on the boundary portion.

6. The light irradiation device according to claim 2, wherein a diameter of the boundary portion continuously decreases from the large diameter portion toward the small diameter portion.

7. The light irradiation device according to claim 2, wherein the tubular body includes an outer layer portion that covers an outer side of the small diameter portion.

8. The light irradiation device according to claim 2, wherein the tubular body includes an outer layer portion that integrally covers outer sides of the small diameter portion, the boundary portion, and the large diameter portion.

9. The light irradiation device according to claim 2, wherein the tubular body includes a deformation absorption portion having an outer diameter smaller than an outer diameter of the large diameter portion at a proximal end portion.

10. A light irradiation device comprising: a balloon formed from a light transmissive material; and a tubular body arranged inside the balloon and including a lumen through which a light irradiation body including a light irradiation unit is inserted, wherein the balloon includes a light transmissive window portion capable of transmitting light from the light irradiation unit and a light shielding body that is disposed in a region other than the light transmissive window portion and has a lower transmittance of light from the light irradiation unit than a transmittance of the light transmissive window portion, the light transmissive window portion including a first end portion and a second end portion, and the lumen of the tubular body includes a boundary portion on which a distal end of the light irradiation body abuts, at a position distal to the first end portion and proximal to a most distal end of the balloon.

11. The light irradiation device according to claim 10, wherein the lumen of the tubular body includes a small diameter portion distal to the boundary portion, and a large diameter portion having an inner diameter larger than an inner diameter of the small diameter portion proximal to the boundary portion.

12. The light irradiation device according to claim 11, wherein the inner diameter of the small diameter portion is smaller than an outer diameter of the light irradiation body.

13. The light irradiation device according to claim 12, wherein the inner diameter of the small diameter portion is larger than an outer diameter of a core wire to be inserted into the lumen.

14. The light irradiation device according to claim 11, wherein a diameter of the boundary portion continuously decreases from the large diameter portion toward the small diameter portion.

15. The light irradiation device according to claim 11, wherein the tubular body includes an outer layer portion that covers an outer side of the small diameter portion.

16. The light irradiation device according to claim 11, wherein the tubular body includes an outer layer portion that integrally covers outer sides of the small diameter portion, the boundary portion, and the large diameter portion.

17. The light irradiation device according to claim 11, wherein the tubular body includes a deformation absorption portion having an outer diameter smaller than an outer diameter of the large diameter portion at a proximal end portion.

18. A method of performing light irradiation treatment, comprising: inserting a light irradiation device into a living body, the light irradiation device comprising a balloon formed from a light transmissive material, and a tubular body arranged inside the balloon and including a lumen through which a light irradiation body including a light irradiation unit is inserted, wherein the lumen of the tubular body includes a boundary portion on which a distal end of the light irradiation body abuts; aligning a light transmissive window portion of the balloon with a portion of the living body to be treated; expanding the balloon; and irradiating the portion of the living body with light transmitted from the light irradiation unit through the window.

19. The method of claim 18, further comprising deflating the balloon after irradiating the portion of the living body.

20. The method of claim 19, further comprising removing the light irradiating device from the living body after deflating the balloon.
